(19) United States
(12) Patent Application Publication
(10) Pub. No.: US 2016/0083684 A1
(43) Pub. Date: Mar. 24, 2016

(54) ETHANOL PRODUCTION DEVICE AND PROCESS THROUGH CONTINUOUS SOLID-STATE FERMENTATION AND HAVING SELF-CONTROL SYSTEM

(71) Applicant: TSINGHUA UNIVERSITY, Beijing (CN)

(72) Inventors: Shizhong Li, Beijing (CN); Lei ZHANG, Beijing (CN); Guangming LI, Beijing (CN)

(21) Appl. No.: 14/958,482

(22) Filed: Dec. 3, 2015

Related U.S. Application Data
(63) Continuation of application No. PCT/CN2014/071580, filed on Jan. 27, 2014.

(30) Foreign Application Priority Data
Aug. 7, 2013 (CN) 201310341882.3

(51) Int. Cl. C12M 1/00; C12P 7/06; C12M 1/34; C12M 3/04; C12M 1/16; C12M 1/26

(52) U.S. Cl. CPC C12M 21/12; C12M 21/16; C12M 33/16; C12M 29/06; C12M 33/18; C12M 41/00; C12M 41/12; C12M 27/10; C12P 7/065

(57) ABSTRACT

The present invention belongs to the technology field of microbial fermentation for producing fuel ethanol. It specifically relates to a device and process having self-control system for producing fuel ethanol. The process mainly comprises the two steps of strain addition and continuous solid-state fermentation, that is, adding the fermentation bacterial strains into crushed raw materials before the crushed materials entering into the continuous solid-state fermentation tank and then the above materials mixed with bacteria are fermented in the continuous solid-state fermentation tank, making the fermentable sugars into ethanol.

### CROSS-REFERENCE TO RELATED APPLICATIONS

This application is a continuation application of International Patent Application No. PCT/CN2014/071580, filed Jan. 27, 2014, which itself claims priority to Chinese Patent Application No. 201310341882.3, filed Aug. 7, 2013, which are incorporated herein by reference in their entireties.

### TECHNICAL FIELD

The present invention belongs to the technology field of fuel ethanol production through microbial fermentation. It specifically relates to a solid-state fermentation device and process for producing fuel ethanol, particularly a self-control continuous solid-state fermentation device and process for producing fuel ethanol from sugar-containing stocks.

### BACKGROUND OF THE INVENTION

With the development of the society, the fossil resource has gradually depleted, countries around the world begin working on renewable energy materials, and a new green energy—fuel ethanol, which can be used to replace fossil fuel, has been widespread concern at home and abroad. Because its combustion characteristics are very close to gasoline, fuel ethanol can be used by being blended with gasoline according to certain proportions, and can also be used directly as a fuel, greatly reducing harmful gas emissions. Fuel ethanol has a broad development space.

As the world population increase rapidly, in order to ensure food supply, some countries gradually begin to imply policies restricting the production ethanol from food crops, thus producing ethanol from non-food crops would be a trend. Sweet sorghum, for its characteristics such as drought resistance, salt and alkali resistance, highly adaptable, and higher sugar content in the stem, becomes a preferred candidate of such non-food crops.

There are mainly two ways of liquid- and solid-state fermentations for producing ethanol from sweet sorghum straws. The liquid-state fermentation is to ferment the juice squeezed from fresh sweet sorghum straws; however, the preservatives added necessarily for the juice storage will adverse to the fermentation yeast, and the squeezed solid materials will contain a certain amount of residual sugar, and both will decrease the ethanol yield. The solid-state fermentation is to directly ferment crushed sweet sorghum straws, which simplifies the raw material pretreatment, avoids the use of preservatives, and reduces sugar loss, thus fundamentally overcoming the defects of liquid fermentation.

However, the most current solid-state fermentation technologies still follow the traditional wine making process, which is carried out in a fixed fermentation pool or fermentation tank, and is operated intermittently, and thus the alcohol is volatile, and the production efficiency is low. Also, the crushed straw material has the characteristics of intertwining, and thus it is difficult to realize the continuous flow, and the uncertainty of its movement also brings to monitoring difficulties. Therefore, the top priority in the development of the industry of producing fuel ethanol through the biomass fermentation is to develop new device, reduce the cost and energy consumption, increase the ethanol yield, and achieve continuous solid-state fermentation.

In the Chinese patent application publication CN 102071222A, the inventors of the present invention disclosed a continuous solid-state fermentation device and process for producing fuel ethanol, wherein the continuous fermentation is accomplished by the following steps:

(a) bacterial strains addition: adding the fermentation bacterial strains into the fermentation materials before the crushed materials entering into the continuous solid-state fermentation tank; the amount of the fermentation inoculation is 5%-20% (V/W);

(b) continuous solid-state fermentation: the above mixed fermentation materials are fermented continuously in the continuous solid-state fermentation tank for a fermentation time of 30 h, the rotation speed of the continuous solid-state fermentation tank is kept at 0.03-0.4 rpm, and the temperature in the continuous solid-state fermentation tank is kept at 25-40° C., making the fermentable sugars into ethanol.

The technical solutions of the self-controlled continuous solid-state fermentation device for producing fuel ethanol disclosed in the China patent application publication CN 102071222A is: a feeder is connected with airtight bin, which is provided with a material level meter and a bacteria liquid spray tube, and is installed a dual roll feeder on the outlet; the outlet of the dual roll feeder is connected with a feeding screw feeder, which stretches into a feeding airtight bin and is fixed, and the feeding airtight bin and the continuous solid-state fermentation tank are mechanically sealed; the continuous solid-state fermentation tank has an inclination angle toward the outlet along the axis direction, and the outside wall of the tank is fixed with ring gears, with which a variable speed motor driving device engagements, driving the continuous solid-state fermentation tank rollover under the support of a carrier roller; the outlet of the continuous solid-state fermentation tank is connected with the discharging airtight bin, on which is provided with an air outlet and installed a breathing valve, and the discharging airtight bin and the continuous solid-state fermentation tank are mechanically sealed. The discharging airtight bin is installed a gate valve on the outlet, and is air-tightly connected with subsequent apparatuses by a gate valve.

The inside wall of the continuous solid-state fermentation tank is installed several groups of distributing plates, which have the number of 6-10 in each group, and have an adjustable installation angle; the distributing plates in the same group are equally spaced on same cross section, and the distributing plates on different cross section are staggered, so as to change the agitating degree of the materials and regulate the flow speed of the materials.

The dual roller feeder and the airtight feeding bin are sealed by material accumulation, and the discharging airtight bin and the gate valve are sealed with the fermentation materials.

The nozzles and connection parts of the device and the joints of the device with other apparatuses at the front and the rear are air-tightly sealed, and adapt to the rotary motion of the fermentation tank, to ensure the effect of air-tightly sealing in the whole operation process.

The rotation speed of the continuous solid-state fermentation tank is adjustable between 0.03 and 0.4 rpm.

However, the above device and process used for continuous fermentation still have some defects, such as: 1. Because that the pulverized straw particles are large, the feeding inlet is often clogged when feeding with a conventional feeder, and often needs to be dredged, thus affecting production efficiency; 2. The fermented straws tie and interweave together, and would clog the discharging outlet, thus also affecting production efficiency; 3. Because that the heat generated during the fermentation will continuously raise the temperature in the fermentation tank, when the temperature is above the suitable range of the bacterial strains, it will affect the fermentation efficiency; 4. Because that the medium for the bacteria liquid is expensive, the production cost of the bacterial strains is high, the used concentration of the added bacterial strain is low, the fermentation efficiency is not optimum, etc.; 5. The fermentation device is not automatically adjusted, thus also affecting the normal production.

Thus, there remains a need in the art to achieve a self-controlled continuous solid-state fermenting device and process. This is to solve the problems of the high pretreatment cost of liquid fermentation materials, the low production efficiency of solid state fermentation and the difficulty in achieving continuity during the current process of producing ethanol from sweet sorghum straws.

### SUMMARY OF THE INVENTION

In order to solve the above technical problems, in view of the flowing characteristics of the materials, combined with the operating characteristics of fermentation process, the present invention provides a self-controlled continuous solid-state fermentation device and process for producing fuel ethanol, so as to realize the continuous production of fuel ethanol from sweet sorghum straws.

In a first aspect of the present invention, there is provided a self-controlled continuous solid-state fermentation device for producing fuel ethanol, the device comprising: a feeder (101), a screw conveyor for feeding (102), a bacteria liquid spray tube (103), an airtight bin for feeding (109), a fermentation tank (105), a screw conveyor for discharging (114), characterized in that: the outlet of the feeder (101) is connected with the screw conveyor for feeding (102), on which is provided with the bacteria liquid spray tube (103); the screw conveyor for feeding (102) protrudes into the feeding airtight bin (109) and is fixed, and the feeding airtight bin (109) and the continuous solid-state fermentation tank (105) are mechanically sealed; the continuous solid-state fermentation tank (105) has an inclination angle toward the outlet along the axis direction, and the outside wall of the tank is fixed with a ring rear (106), with which a variable speed motor driving device (112) engagements, driving the continuous solid-state fermentation tank (105) to roll over under the support of a carrier roller (110) at an adjustable rotation speed between 0.03 and 0.4 rpm. The inside wall of the continuous solid-state fermentation tank (105) is installed several groups of distributing plates, which have the number of 11-15 in each group, and have an adjustable installation angle, so as to change the agitating degree of the materials and regulate the flow speed of the materials; the distributing plates in the same group are equally spaced on the same cross section, and the distributing plates on different cross sections are staggered, so as to change the agitating degree of the materials and regulate the flow speed of the materials. The outlet of the continuous solid-state fermentation tank (105) is connected with the screw conveyor for discharging (114), on which is provided with an air outlet (107) and installed a breathing valve (108), and the screw conveyor for discharging (114) and the continuous solid-state fermentation tank (105) are mechanically sealed, and is air-tightly connected with the subsequent apparatuses.

In an embodiment of the present invention, the fermentation tank (105) has an inclination angle of 1-5° with the horizontal plane, and the feeding inlet is higher than the discharging outlet.

In an embodiment of the present invention, the fermentation tank (105) has plurality of externally mounted temperature measuring ports, forming temperature measuring port set (111), to monitor the temperature in the fermentation tank.

In an embodiment of the present invention, the inside wall of the continuous solid-state fermentation tank (105) is installed several groups of distributing plates, which have the number of 11-15 in each group; the distributing plates in the same group are equally spaced on the same section, and the distributing plates on the different sections are staggered, so as to change the agitating degree of the materials and regulate the flow speed of the materials.

In an embodiment of the present invention, the nozzles and connection parts of the device and the joint of the device with other apparatuses at the front and rear are air-tightly sealed, and are adapt to the rotary motion of the fermentation tank, to ensure the effect of air-tightly sealing in the whole operation process.

In an embodiment of the present invention, the rotation speed of the continuous solid-state fermentation tank is adjustable between 0.03 and 0.4 rpm.

On another aspect of the present invention, there is provided a process of continuous solid-state fermentation for producing fuel ethanol, comprising the following steps:

(a) bacterial strains addition: adding the fermentation bacterial strains into the fermentation materials before the crushed materials entering into a continuous solid-state fermentation tank; the amount of the fermentation inoculation is 5%-20% (V/W); the concentration of the bacterial inoculations is 1-9×10⁸/ml;

(b) continuous solid-state fermentation: the above mixed materials are fermented continuously in the continuous solid-state fermentation tank for a fermentation time of 24-40 h, the rotation speed of the continuous solid-state fermentation tank is kept at 0.03-0.4 rpm, and the temperature in the continuous solid-state fermentation tank is kept at 25-40° C., making the fermentable sugars into ethanol;

In an embodiment of the present invention, the procedure of bacterial strains addition in the step (a) is done in the screw conveyor for feeding before the materials entering into the continuous solid-state fermentation tank.

In an embodiment of the present invention, in the step (b), the rotary motion of the continuous solid-state fermentation tank itself realizes the uniform mixing of materials and theirs continuous movement to the outlet.

In an embodiment of the present invention, the bacterial strain used in the fermentation is CGMCC1949.

The device and process of the continuous solid-state fermentation of the present invention have the following beneficial effects:

(1) The improved design of the feeding inlet and the discharging outlet of the continuous solid-state fermentation tank, coordinating with the appropriate inclination angle and the rotation speed of the fermentation tank, makes the materials to move steadily and smoothly, which really realizes the continuity of the solid-state fermentation process, and omits the step of unloading and shipping to the next apparatus by hand, improving the utilization rate of the fermentation tank, thus effectively improving the production efficiency, and saving manpower and reducing labor intensity, and thus to achieve the goal of reducing the energy consumption and production cost.

(2) The continuous fermentation is carried out in an airtight system, and the materials directly enter into the next unit after the completion of the fermentation, the operation process having no intermittence and the materials being unexposed to air; also, the designed vent plays the role of controlling pressure in the tank, and maintain a little positive pressure, to ensure the anaerobic environment of the bacterial strains during fermentation, thus benefiting to the fermentation.

(3) The angle of the distributing plates in the fermentation tank can be flexibly adjusted, can change the agitating degree of the materials and regulate the flow speed of the materials, so as to meet the needs of different amount of heat releasing in the different stages of the fermentation, to ensure the normal fermentation process, and further to have some regulating effect on the production.

(4) The device can change the rotation speed of the fermentation tank through adjusting the motor speed, making its production capacity adjustable within a certain range, further realizing the controllability of the production.

(5) The use of the solid-state fermentation reduces the intensity of the pretreatment of the raw materials, and saves time and manpower; the full usability of the fermentable sugars in sweet sorghum straws and no use of a preservative reduce sugar loss, ensure the activity of bacteria growth, and improve product quality and yield.

### BRIEF DESCRIPTION OF DRAWINGS

FIG. 1 is a structural schematic of the self-controlled continuous solid-state fermentation device of the present invention. The appended drawing reference signs in the figure: 101—a feeder; 102—a screw conveyor for feeding; 103—a bacteria liquid spray tube; 104—a steam nozzle; 105—a continuous solid-state fermentation tank; 106—a gear ring; 107—an air outlet; 108—a breathing valve; 109—a feeding airtight bin; 110—a carrier roller; 111—a temperature measuring port set; 112—a variable speed motor driving device; 113—a thermal insulating layer; 114—a screw conveyor for discharging.

FIG. 2 is the distribution schematic of the distributing plates on any distributing plate section of the fermentation tank as shown in FIG. 1.

### DETAILED DESCRIPTION OF THE INVENTION

The present invention provides a continuous solid-state fermentation device and process for producing fuel ethanol, to solve the problems of the high pre-treating cost of the liquid fermentation materials, the low production efficiency of solid state fermentation and the difficulty in achieving continuity during the current process of producing ethanol from sweet sorghum straws, realizing continuous production of fuel ethanol from sweet sorghum straws.

In the fermentation device of the present invention, due to the cancellation of feeding hopper and the addition of a screw conveyor for discharging at the discharging outlet, the following problems are avoided, such as: 1. Because that the pulverized straw particles tie and interweave together, and when feeding with a conventional feeder, would often clog the feeding inlet, which often needs to be dredged, thus affecting production efficiency; 2. The fibers of the fermented straws after interweave together, and is very likely to clog the discharging outlet, also affecting the production efficiency.

In addition, the bacterial strain used in the fermentation process of the present invention is CGMCC1949, which can grow well on the corn medium, thus avoiding the use of traditional wort medium, reducing production cost, increasing the concentration of bacterial strain, and further improving fermentation efficiency.

The present invention will now be further explained by combining with the appended drawings.

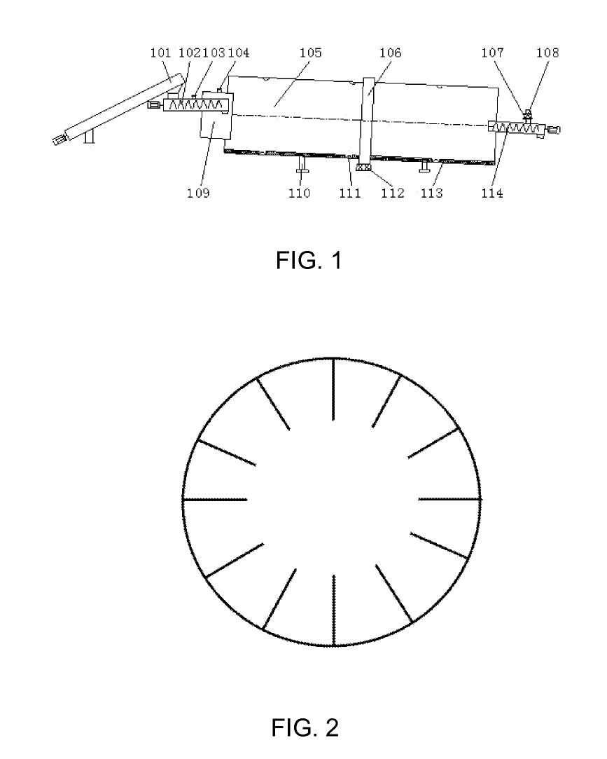

FIG. 1 is a structural schematic of the self-control continuous solid-state fermentation device of the present invention. As shown in FIG. 1, the outlet of the feeder 101 is connected with the screw conveyor for feeding 102, on which is provided with the bacteria liquid spray tube 103; the screw conveyor for feeding 102 protrudes into the feeding airtight bin 109 and is fixed, the feeding airtight bin 109 and the continuous solid-state fermentation tank 105 are mechanically sealed; the continuous solid-state fermentation tank 105 has an inclination angle toward the outlet along the axis direction, and the outside wall of the tank is fixed with the ring rear 106, with which the variable speed motor driving device 112 engagements, driving the continuous solid-state fermentation tank 105 to roll over under the support of the carrier roller 110 at an adjustable rotation speed between 0.03 and 0.4 rpm. The inside wall of the continuous solid-state fermentation tank 105 is installed several groups of distributing plates, which have the number of 11-15 in each group, and have an adjustable installation angle, so as to change the agitating degree of the materials and regulate the flow speed of the materials; the distributing plates in the same group are equally spaced on the same section, and the distributing plates on different section are staggered, so as to change the agitating degree of the materials and regulate the flow speed of the materials. The outlet of the continuous solid-state fermentation tank 105 is connected with the screw conveyor for discharging 114, on which is provided with the air outlet 107 and installed the breathing valve 108, and the screw conveyor for discharging 114 and the continuous solid-state fermentation tank 105 are mechanically sealed, and is air-tightly connected with subsequent apparatuses.

The continuous solid-state fermentation tank of the present invention has plurality of externally mounted temperature measuring ports, forming temperature measuring port set 111, to monitor the temperature in the fermentation tank, the temperature in the fermentation tank should be keep at 25-40° C., and when the fermentation temperature is too high, a method for cooling such as external water spray or air cooling would start. If used in a cold area or season, the continuous solid-state fermentation tank of the present invention is provided with an outside thermal insulation layer. This would ensure the stability of the fermentation temperature, and also realize self-controlling of the temperature.

Before continuous feeding, steam should be input from the steam nozzle on the fermentation tank for sterilization disinfection. During fermenting, crushed sweet sorghum straws are fed into the screw conveyor for discharging 102 by the feeder 101, and mixed with the bacteria liquid inputted by the bacteria liquid spray tube 103, and the mixed materials enter into the fermentation tank 105 via the screw conveyor for discharging 102; the variable speed motor driving device 112 engagements with the gear rings 106, thus driving the continuous solid-state fermentation tank 105 to roll over under the support of the carrier roller 110 at a certain speed, and the sweet sorghum straw materials, under the effects of its own gravity and the distributing plates, move to the outlet of the fermentation tank along the inclined tank, and then are discharged continuously via the screw conveyor for discharging 114. The materials complete the continuous fermentation during flowing, and the fermented materials enter into an ethanol distillation process. The fermentation tank has an outside segment-dismountable thermal insulation layer 113 and a water spray or air cooling apparatus, so that the different amount of heat releasing in the fermentation decides whether to thermally insulate or cool the tank. The mixed gases formed by carbon dioxide and ethanol gas produced in fermentation together with water vapor discharge from the air outlet 107 and enter into a mixed gas processing apparatus, for the recovery of carbon dioxide and ethanol gas.

FIG. 2 is the distribution schematic of the distributing plates on any distributing plate section of the fermentation tank as shown in FIG. 1.

### EXAMLES

### Example 1

(Continuous Fermentation)

After mature sweet sorghum straws were harvested, the sweet sorghum straws that had 70% of water content, 11% of total sugar content, and 7% of reducing sugar content were used as the raw materials, and were crushed into threads with diameter of 1-2 mm and length less than 30 mm. In the meantime, the bacterial strain CGMCC1949 was added in the screw conveyor for feeding, which was mixed with the crushed materials and the mixture were fermented continuously in the continuous solid-state fermentation tank. The amount of the fermentation inoculation was 10% (V/W), and various operation parameters were adjusted, to keep a temperature of 25-40° C. in the tank, a fermentation time of 24 hours, and a rotation speed of 0.25 rmp of the fermentation tank. After fermentation, the measured ethanol yield was 91.2%, and the measured total sugar conversion rate was 94.2%.

### Example 2

(Continuous Fermentation)

After the mature sweet sorghum straws were harvested, the sweet sorghum straws that had 70% of water content, 12% of total sugar content, and 8% of reducing sugar content were used as the raw material, and were crushed into threads with diameter of 1-2 mm and length of less than 30 mm. In the meantime, the bacterial strain CGMCC1949 was added in the screw conveyor for feeding, which was mixed with the crushed materials and the mixture were fermented continuously in the continuous solid-state fermentation tank. The amount of the fermentation inoculation was 15% (V/W), and various operation parameters were adjusted, to keep a temperature of 25-40° C. in the tank, a fermentation time of 30 hours, and a rotation speed of 0.1 rmp of the fermentation tank. After fermentation, the measured ethanol yield was 92.3%, and the measured total sugar conversion rate was 96.4%.

What is claimed is:

1. A self-controlled continuous solid-state fermentation device for producing fuel ethanol, the device comprising: a feeder (101), a screw conveyor for feeding (102), a bacteria liquid spray tube (103), an airtight bin for feeding (109), a fermentation tank (105), a screw conveyor for discharging (114), characterized in that the outlet of the feeder (101) is connected with the input of the screw conveyor for feeding (102), on which is provided with the bacteria liquid spray tube (103); the screw conveyor for feeding (102) protrudes into the feeding airtight bin (109) and is fixed, and the feeding airtight bin (109) and the continuous solid-state fermentation tank (105) are mechanically sealed; the continuous solid-state fermentation tank (105) has an inclination angle toward the outlet along the axis direction, and the outside wall of the tank is fixed with a ring rear (106), with which a variable speed motor driving device (112) engagements, driving the continuous solid-state fermentation tank (105) to roll over under the support of a carrier roller (110) at an adjustable rotation speed between 0.03 and 0.4 rpm. The inside wall of the continuous solid-state fermentation tank (105) is installed several groups of distributing plates, which have the number of 11-15 in each group, and have an adjustable installation angle, so as to change the agitating degree of the materials and regulate the flow speed of the materials; the distributing plates in the same group are equally spaced on the same cross section, and the distributing plates on different sections are staggered, so as to change the agitating degree of the materials and regulate the flow speed of the materials. The outlet of the continuous solid-state fermentation tank (105) is connected with the screw conveyor for discharging (114), on which is provided with an air outlet (107) and installed a breathing valve (108), and the screw conveyor for discharging (114) and the continuous solid-state fermentation tank (105) are mechanically sealed, and is air-tightly connected with subsequent apparatuses.

2. The self-controlled continuous solid-state fermentation device for producing fuel ethanol as claimed in claim 1, characterized in that the fermentation tank (105) has an inclination angle of 1-5° with the horizontal plane, and the feeding inlet is higher than the discharging outlet.

3. The self-controlled continuous solid-state fermentation device for producing fuel ethanol as claimed in claim 1, characterized in that the fermentation tank (105) has plurality of externally temperature measuring ports on the tube across the diameter at different sections, forming temperature measuring port set (111), to monitor the temperature in the fermentation tank.

4. The self-controlled continuous solid-state fermentation device of self-control for producing fuel ethanol as claimed in claim 1, characterized in that the inside wall of the continuous solid-state fermentation tank (105) is installed several groups of distributing plates, which have the number of (1-5) D (D is the diameter of the fermentation tank, and the unit is m,) in each group; the distributing plates in the same group are equally spaced on the same section, and the distributing plates on different sections are staggered, so as to change the agitating degree of the materials and regulate the flow speed of the materials.

5. The self-controlled continuous solid-state fermentation device of self-control for producing fuel ethanol as claimed in claim 1, characterized in that the nozzles and connection parts of the device and the joint of the device with other apparatuses at the front and rear are air-tightly sealed, and are adapt to the rotary motion of the fermentation tank, to ensure the effect of air-tightly sealing in the whole operation process.

6. The self-controlled continuous solid-state fermentation device of self-control for producing fuel ethanol as claimed in claim 1, characterized in that the rotation speed of the continuous solid-state fermentation tank (105) is adjustable between 0.03 and 0.4 rpm.

7. A continuous solid-state fermentation process for producing fuel ethanol, comprising the following steps:
(a) bacterial strains addition: adding the fermentation bacterial strains into the fermentation materials before the crushed materials entering into a continuous solid-state fermentation tank; the amount of the fermentation inoculation is 5%-20% (V/W); and the concentration of the bacterial inoculations is 1-9×10⁸/ml;
(b) continuous solid-state fermentation: the above mixed materials are fermented continuously in the continuous solid-state fermentation tank for a fermentation time of 24-30 h, the rotation speed of the continuous solid-state fermentation tank is kept at 0.03-0.4 rpm, and the temperature in the continuous solid-state fermentation tank is kept at 25-40° C., making the fermentable sugars into ethanol.

8. The continuous solid-state fermentation process for producing fuel ethanol as claimed in claim 7, characterized in that, the procedure of bacterial strains addition in the step (a) is done in the airtight screw conveyor for feeding before the materials entering into the continuous solid-state fermentation tank.

9. The continuous solid-state fermentation process for producing fuel ethanol as claimed in claim 7, characterized in that, in the step (b), the rotary motion of the continuous solid-state fermentation tank itself realizes the uniform mixing of the materials and theirs continuous movement to the outlet.

10. The continuous solid-state fermentation process for producing fuel ethanol from sugar raw materials as claimed in claim 7, characterized in that, the bacterial strain used in the fermentation is CGMCC1949.
